(19) United States
(12) Patent Application Publication
(10) Pub. No.: US 2018/0002210 A1
(43) Pub. Date: Jan. 4, 2018

(54) BIOMINERALOGICAL METHOD AND APPARATUS FOR REMOVING CESIUM IONS

(71) Applicant: KOREA ATOMIC ENERGY RESEARCH INSTITUTE, Daejeon (KR)

(72) Inventors: Seung Yeop LEE, Daejeon (KR); Jin Ha HWANG, Ulsan (KR); Min-Hoon BAIK, Daejeon (KR); Bum Kyoung SEO, Daejeon (KR); Minhee LEE, Busan (KR)

(21) Appl. No.: 15/471,551

(22) Filed: Mar. 28, 2017

(30) Foreign Application Priority Data
Jul. 4, 2016 (KR) 10-2016-0084011

(51) Int. Cl. C02F 3/34; C12M 1/00; C12P 3/00; C02F 1/52; C01G 49/00; C02F 103/08; C02F 101/00; C02F 101/10

(52) U.S. Cl. CPC C02F 3/34; C02F 1/5236; C02F 3/345; C12M 47/10; C12M 29/14; C01G 49/009; C12P 3/00; C12M 47/12; C02F 2101/006; C02F 2101/10; C02F 2103/08

(57) ABSTRACT

Provided are a biomineralogical method for removing cesium ions. The method for removing cesium ions, the method comprising: adding metal-reducing bacteria, an iron source, and a sulfur source into a solution containing the cesium ions to convert the cesium ions into a solid mineral incorporating cesium. The method for removing cesium ions according to the present invention has advantages in that the cesium ions may be removed with high efficiency and small volume even in the case in which competing ions are present at a high concentration like sea water.

### CROSS-REFERENCE TO RELATED APPLICATIONS

This application claims priority under 35 U.S.C. §119 to Korean Patent Application No. 10-2016-0084011, filed on Jul. 4, 2016, in the Korean Intellectual Property Office, the disclosure of which is incorporated herein by reference in its entirety.

### TECHNICAL FIELD

The following disclosure relates to a biomineralogical method and an apparatus for removing cesium ions from a wastewater with cesium.

### BACKGROUND

Examples of radioactive nuclides mainly emitted in a case in which a severe accident occurs in a nuclear facility such as a nuclear power plant include Co-60, Cs-137, and the like. In particular, since Cs-137, radioactive cesium, has a long half-life (about 30 years), a technology capable of highly efficiently removing or separating the radioactive cesium in a short time has been required.

As an example, a large amount of radioactive nuclide, particularly, radioactive cesium was leaked to fresh water or sea water due to leakage of radioactive matters from Fukushima nuclear power plant in 2011. A necessity for a technology capable of highly efficiently removing the radioactive cesium leaked to fresh water or sea water as described above has increased, and various researchers have conducted research for separating and removing radioactive cesium.

Currently, a widely known method for removing cesium is mainly to use an adsorbent such as zeolite, or the like. These adsorbents may remove cesium with high efficiency under a high-concentration and competing ion-free condition, but in a case in which a large number of competing ions are present and a concentration of cesium is excessively low, efficiency may be significantly decreased. As an example, a cesium adsorbent selectively adsorbing and separating cesium has been disclosed in Korean Patent Laid-Open Publication No. 10-2015-0137201, but in the case of using this adsorbent, a large amount of wastes containing the adsorbent may be generated, and in a state in which a competing ion is present, efficiency may be significantly decreased.

As described above, the leaked radioactive cesium has been mainly introduced into sea water cooling heat of a nuclear reactor, and a technology for removing cesium dissolved in sea water is particularly required. However, radioactivity of the radioactive cesium as described above is significantly high, but a concentration thereof is excessively low (at most 0.5 ppm or less; for example, highly contaminated water in Fukushima), such that it is significantly difficult to remove the radioactive cesium. Particularly, in the case of sea water, a large number of competing cations such as sodium ions, potassium ions, and the like, are present, such that in order to remove cesium having an excessively low concentration, a more advanced technology is required.

### RELATED ART DOCUMENT

### Patent Document

### Korean Patent Laid-Open Publication No. 10-2015-0137201

### SUMMARY

The present invention is to solve the above-mentioned problems.

An embodiment of the present invention is directed to providing a method and an apparatus for efficiently removing a large amount of cesium ions at room temperature.

Another embodiment of the present invention is directed to providing a method and an apparatus for highly efficiently removing cesium ions even at a low concentration with high radioactivity (for instance, Cs-137).

Another embodiment of the present invention is directed to providing a method and an apparatus for efficiently removing cesium ions even at a state in which competing ions are present at high concentrations just like sea water.

Another embodiment of the present invention is directed to providing a method and an apparatus for removing cesium ions capable of biomineralizing cesium at room temperature to significantly decrease a volume of waste in a compact solid form.

Another embodiment of the present invention is directed to providing a method and an apparatus for removing cesium ions by biomineralizing them to maintain wastes in a stable solid form for a long period of time at the time of underground disposal.

The present invention provides a method for removing cesium ion capable of solving the above-mentioned problems.

In one general aspect, a method for removing cesium ion includes mixing metal-reducing bacteria, an iron source, and a sulfur source with a solution containing the cesium ions to convert the cesium ions into a mineral form containing cesium.

The mineral containing cesium may be Pautovite (CeFe₂S₃).

In the converting of the cesium ions into the mineral form containing cesium, a pH of the solution may be 7 to 8.5.

The metal-reducing bacteria may be one or two or more selected from the group consisting of Pseudomonas, Shewanella, Clostridium, Desulfovibrio, Desulfosporosinus, Desulfotomaculum, Anaeromyxobacter, and Geobacter.

A concentration of the metal-reducing bacteria (based on a protein concentration) may be 0.3 to 5 mg/L.

The iron source may be one or two or more selected from iron (II) chloride, iron (II) sulfate, iron (II) acetate, iron (II) bromide, and iron (II) nitride.

A concentration of the iron source may be 0.5 to 5 mM.

The sulfur source may be a compound forming anions represented by SO₄²⁻, SO₃²⁻, SO₂²⁻, S₂O₃²⁻, S₂O₄²⁻, S₂O₅²⁻, S₂O₆²⁻, S₂O₇²⁻, S₂O₈²⁻, S₄O₇²⁻, or S₄O₆²⁻.

The sulfur source may be a dissolved oxygen scavenger.

A concentration of the sulfur source may be 0.3 to 2.0 mM.

Electron donors may be additionally provided for the solution with sulfur source and bacteria.

A concentration of cesium in the solution containing cesium may be 0.5 ppm or less.

The solution containing the cesium ions may be sea water.

In another general aspect, an apparatus for removing cesium ions includes:

an anaerobic tank into which a solution containing cesium ions is introduced and to which a sulfur source and a pH adjustment reagent are supplied; and

a microbial purification tank which is in connection with the anaerobic tank and to which metal-reducing bacteria, an iron source, and an electron donor are supplied,

wherein cesium ions are converted into a crystalline mineral form incorporating cesium by the activity of metal-reducing bacteria to thereby be precipitated from the microbial purification tank, such that the cesium ions in the solution containing cesium ions are removed in a compact form of stable solid sludge.

The apparatus for removing cesium ions may further include:

a first transfer pipe connecting the anaerobic tank and the microbial purification tank so as to be openable and closable;

a first transfer pump connected to the first transfer pipe to transfer the solution containing cesium ions in the anaerobic tank to the microbial purification tank;

a sludge discharge pipe installed so as to be in connection with a lower portion of the microbial purification tank and be openable and closable; and

a sludge discharge pump connected to the sludge discharge pipe to discharge the sludge of the microbial purification tank.

### BRIEF DESCRIPTION OF THE DRAWINGS

FIG. 1 is a schematic view illustrating an apparatus for removing cesium ions according to an exemplary embodiment of the present invention.

FIG. 2 is a schematic view illustrating an apparatus for removing cesium ions according to another exemplary embodiment of the present invention.

FIG. 3 is characteristic curve of cesium removal from sea showing an unusual increase of cesium removal efficiency toward lower cesium concentrations.

FIG. 4 is a characteristic curve of cesium removal from fresh water showing an unusual increase of cesium removal efficiency toward lower cesium concentrations.

FIG. 5 shows curves of cesium removal from fresh water at different cesium concentrations with time.

FIG. 6 shows an electron microscope photograph obtained by observing a crystalline solid mineral composed of cesium, iron and sulfur that were formed according to an exemplary embodiment of the present invention and an element analysis result thereof.

FIG. 7 shows characteristic X-ray diffraction (XRD) patterns for Mackinawite with majority, and Pautovite with minority, which is a crystalline mineral form with a (hkl) index of (221), generated according to an exemplary embodiment of the present invention.

### DETAILED DESCRIPTION OF MAIN ELEMENTS

110: anaerobic tank
120: microbial purification tank
111: sulfur source storage tank
112: pH adjustment reagent storage tank
121: iron source storage tank
122: metal-reducing bacteria storage tank
123: electron donor storage tank
10: first transfer pipe
20: first pump
30: sludge discharge pipe
40: sludge discharge pump
50: purified water discharge pipe
60: purified water discharge pump
70: cesium ion-containing solution inflow pipe
200: control part

### DETAILED DESCRIPTION OF EMBODIMENTS

Hereinafter, a method and an apparatus for removing cesium ions according to the present invention will be described in detail with reference to the accompanying drawings. The following accompanying drawings are provided by way of example so that the idea of the present invention can be sufficiently transferred to those skilled in the art to which the present invention pertains. Therefore, the present invention is not limited to the drawings to be provided below, but may be modified in different forms. In addition, the drawings to be provided below may be exaggerated in order to clarify the scope of the present invention. Here, technical terms and scientific terms used in the present specification have the general meaning understood by those skilled in the art to which the present invention pertains unless otherwise defined, and a description for the known function and configuration unnecessarily obscuring the gist of the present invention will be omitted in the following description and the accompanying drawings.

In a method for removing cesium ions known in the art, which is a method for adsorbing cesium using an adsorbent material, it is difficult to separate and remove only cesium with high efficiency in a state in which competing ions are largely present, and cesium ions are at very low concentrations but have high radioactivity. Therefore, the present applicant conducted research into a method for selectively mineralizing only cesium ions to remove the cesium ions with high efficiency in a state in which competing ions are largely present (for example, sea water conditions), and cesium ions are at very low concentrations but have high radioactivity.

As a result of the research, the present applicant found that the cesium ions may be selectively converted into a solid mineral incorporating cesium along with iron and sulfur by using metal-reducing bacteria in a solution containing cesium ions, and in this case, the cesium ions may be selectively removed even in a state in which competing ions are largely present, and particularly, low-concentration cesium may be effectively removed even under a sea water condition, thereby applying the present invention.

Therefore, the present invention provides a method for removing cesium ions.

The method for removing cesium ions according to the present invention includes:

adding metal-reducing bacteria, an iron source, and a sulfur source with a solution containing the cesium ions to convert the cesium ions into a cesium bearing mineral.

In the case of removing the cesium ions using the method for removing cesium ions according to the present invention, the cesium ions in the solution may be removed through a simple process, cesium ions may be selectively removed even in a state in which the competing ions are largely present, and the cesium ions may be removed with high efficiency even in the case in which a concentration of cesium is very low under a sea water condition. Further, since the cesium ions are converted into the mineral phase containing cesium, long-term disposal stability may be excellent unlike the case of using the general adsorbents that have a problem of cesium desorption, or the like, and a volume of wastes may be significantly decreased, such that disposal cost of the wastes may be significantly decreased. In addition, since there is no need to use the high-cost adsorbents, or the like, the disposal cost may be significantly reduced.

In the method for removing cesium ions according to an exemplary embodiment of the present invention, the mineral containing cesium may be Pautovite (CsFe₂S₃). In the case of mineralizing cesium to isolate cesium as described above, since only the mineral incorporating cesium is removed as a compact sludge, the volume of the wastes may be significantly decreased, and cesium ions may be removed as a higher stable crystalline mineral phase, such that disposal stability may be largely improved. More specifically, in the case of removing cesium ions using an existing method for adsorbing cesium, a volume of wastes including sludge may be significantly large due to a the initial volume of adsorbing materials, which may also increase disposal cost of the wastes. However, in the method for removing cesium ions according to the present invention, since cesium ions are selectively removed as a compact crystalline mineral, the volume of the wastes may be reduced by at most 90% or less as compared to the case of using the general adsorbing materials. As a result, the disposal cost of the waste may be significantly reduced.

In the method for removing cesium ions according to the exemplary embodiment of the present invention, the cesium ions may be effectively removed even in the case of the very low-concentration of cesium under the sea water condition. In the present invention, the term “low-concentration” means that the concentration of cesium ions is 0.5 ppm or less. In the method for removing cesium ions according to the exemplary embodiment of the present invention, the concentration of the cesium ions in the solution containing cesium ions may be 0.5 ppm or less, preferably 0.3 ppm or less, and more preferably, 0.1 ppm or less. The method for removing cesium ions according to the exemplary embodiment of the present invention has a significantly unique feature and an advanced technique by which the lower the concentration of the cesium ions in the solution can be effectively removed unlike the previous methods to adsorb cesium.

More specifically, in the method for removing cesium ions according to the exemplary embodiment of the present invention, in a case in which the concentration of the cesium ions in the solution containing cesium ions is 0.5 ppm or less, cesium removal efficiency may be improved by about three times as compared to a case in which the concentration of the cesium ions is 10 ppm. Further, in the case in which the concentration of the cesium ions is 0.01 ppm or less, the cesium ion removal efficiency may reach at most 99%.

This advantage is particularly useful in the case of removing cesium ions in radioactive waste water under a highly difficult sea water condition using the method for removing cesium ions according to the present invention. This means that considering that a concentration of cesium in discharged radioactive waste water in general is actually 0.2 ppm or less, specifically 0.1 ppm or less, significantly low-concentration cesium ions may be more efficiently removed in the radioactive waste water under the actually discharged sea water condition (for instance, Fukushima reactor).

In the method for removing cesium ions according to the exemplary embodiment of the present invention, the solution containing cesium ions may be a solution containing the cesium ions and competing ions. Here, the competing ions mean some cations except for the cesium ion, specifically, metal cations except for cesium, and more specifically, alkali metal ions, alkali earth metal ions, or the like. In this case, the alkali metal ions may be a lithium ion, a sodium ion, a potassium ion, or a rubidium ion, and the alkali earth metal ions may be a magnesium ion, a calcium ion, a strontium ion, or a barium ion. Here, a representative example of the solution containing the competing ions may be fresh water or sea water contaminated with radioactive cesium, specifically, sea water contaminated with radioactive cesium.

The method for removing cesium ions according to the exemplary embodiment of the present invention has an advantage in that cesium ions may be efficiently removed even in the case in which the competing ions are largely present. Generally, in the case of removing cesium ions in a solution using the common adsorbents, or the like, when the competing ions are present, the competing ions may be preferably adsorbed instead of the cesium ion due to the very lower concentration of cesium, such that cesium ion removal efficiency may be significantly decreased. Therefore, there has never been known for a case in which low-concentration cesium ions (0.01 ppm or less) are removed with efficiency of 90% or more in the state in which the competing ions are present, specifically, under the sea water condition. However, the method for removing cesium ions according to the exemplary embodiment of the present invention has a superiority in that the cesium ions are selectively mineralized in a form of a crystal phase incorporating cesium, and thus, even though other competing ions are present, there is almost no influence of other cations, and only low-concentration cesium ions may be selectively removed, whereby only the cesium ions may be removed with efficiency of 90% or more, even in a state in which the competing ions are present.

The advantage as described above is particularly useful in the case of actually using the method according to the present invention to remove radioactive waste water. More specifically, in the case of removing radioactive cesium using the common adsorbents, or the like, when the radioactive waste water is sea water, concentrations of competing ions such as sodium ions, calcium ions, magnesium ions, and the like, which are present in the radioactive waste water are several thousands to several ten thousands times higher than the concentration of radioactive cesium (generally, in the case of Na, a concentration is 10,000 ppm or more under the sea water condition). These competing ions significantly interfere with the selectivity for cesium ions, thereby allowing the adsorption of cesium ions to become insignificant. On the contrary, the method for removing cesium ions according to the exemplary embodiment of the present invention is more advantageous in removing only cesium by its selective mineralization, even a low concentration of cesium, such that even though a large number of competing ions are present, the cesium ions may be efficiently removed. This unique feature is significantly useful for purifying waste water containing radioactive cesium that is actually at very low concentration but has high radioactivity for instance, sea water contaminated with radioactive cesium.

In the method for removing cesium ions according to the exemplary embodiment of the present invention, the metal-reducing bacteria are not particularly limited as long as the metal-reducing bacteria are bacteria reducing a sulfur source to be described below. In detail, the metal-reducing bacteria may be one or two or more selected from the group consisting of Pseudomonas, Shewanella, Clostridium, Desulfovibrio, Desulfosporosinus, Desulfotomaculum, Anaeromyxobacter, and Geobacter.

The metal-reducing bacteria according to the present invention may reduce a sulfur source (sulfur oxyanions) to be described below to form S²⁻ (sulfide). While conducting research into the method for removing cesium ions, the present inventor found that when iron (II) ions and cesium ions are present in a solution in which S²⁻ is formed by the above-mentioned metal-reducing bacteria, S²⁻, the iron (II) ions, and the cesium ions react with each other to form a sulfide mineral of Pautovite (CsFe₂S₃) at room temperature, into which the cesium ions may be easily incorporated. In the method for removing cesium ions according to the exemplary embodiment of the present invention, a concentration of the metal-reducing bacteria is not limited as long as the sulfur source may be sufficiently reduced at the concentration, but the concentration (based on a protein concentration) may be 0.3 to 5 mg/L, preferably, 0.5 to 4 mg/L. In the case in which the metal-reducing bacteria are added in the above-mentioned concentration range, the sulfur source (sulfur oxyanions) may be sufficiently reduced to sulfide phase.

In the method for removing cesium ions according to the exemplary embodiment of the present invention, the iron source may be bound to the above-mentioned S²⁻ and cesium ions to thereby be mineralized into a crystalline mineral phase, specifically, Pautovite (CsFe₂S₃) incorporating cesium. Here, as the iron source, any iron reagents may be used without limitation as long as it may provide divalent iron ions (Fe²⁻) to the solution. In detail, the iron source may be one or two or more selected from iron (II) chloride, iron (II) sulfate, iron (II) acetate, iron (II) bromide, and iron (II) nitride.

In the method for removing cesium ions according to the exemplary embodiment of the present invention, concentrations of the iron source are not particularly limited as long as the cesium ions may be sufficiently converted into the iron mineral containing cesium at the concentration. More specifically, the concentration of the iron source may be 0.5 to 5 mM, preferably, 0.1 to 2 mM, but is not limited thereto. In the case in which the concentration of the iron source is in the above-mentioned range, the cesium ions may be readily converted into the cesium-bearing mineral and the above-mentioned metal-reducing bacteria are not affected by an excessive amount of iron ions.

In the method for removing cesium ions according to the exemplary embodiment of the present invention, as the sulfur source, any sulfur reagents (sulfur oxyanions) may be used without limitation as long as it is reduced by the metal-reducing bacteria to form S²⁻ (sulfide) during a process of forming the sulfide mineral containing cesium. In detail, the sulfur source may be a compound forming anions (hereinafter, sulfur oxyanions) represented by SO₄²⁻, SO₃²⁻, SO₂²⁻, S₂O₃²⁻, S₂O₄²⁻, S₂O₅²⁻, S₂O₆²⁻, S₂O₇²⁻, S₂O₈²⁻, S₄O₇²⁻, or S₄O₆²⁻ in a solution. In more detail, the sulfur source may be reagents with cations such as a hydrogen ion, lithium ion, a sodium ion, a potassium ion, calcium ion, a magnesium ion, or the like, together with the above-mentioned anions, but is not limited thereto. As a specific example, the sulfur source may be Na₂SO₃, NaHSO₃, or Na₂SO₄, and as a more specific example, the sulfur source may be Na₂SO₃ or NaHSO₃.

The sulfur source is Na₂SO₃ or NaHSO₃, which is advantageous in that SO₃²⁻ in the solution may react with dissolved oxygen to become SO₄²⁻. That is, the sulfur source may serve as a dissolved oxygen scavenger while serving to supply sulfur to the solution. Oxygen dissolved in the solution is removed by the above-mentioned reaction, which is advantageous for survival of the above-mentioned metal-reducing bacteria, and formation of the mineral containing cesium may be promoted by reducing sulfur oxyanions to sulfide form. As a result, there is an advantage in that cesium ion removal efficiency may be improved.

The concentration of the sulfur source according to the exemplary embodiment of the present invention is not limited as long as the cesium ion may be converted into the cesium-bearing mineral at the concentration, but the concentration of the sulfur source may be specifically, 0.3 to 2.0 mM, and preferably, 0.5 to 1.5 mM. In the case in which the concentration of the sulfur source is in the above-mentioned range, there is an advantage in that the sulfur source may be reduced by the metal-reducing bacteria to sufficiently convert the cesium ions into the cesium-bearing sulfide mineral and at the same time, it is possible to prevent a secondary contamination problem of purified water by an excessive amount of the sulfur source.

In the method for removing cesium ions according to the exemplary embodiment of the present invention, electron donors may be additionally provided into the solution containing the cesium ions. The electron donor may provide electrons required for the sulfur oxyanions reduction by the metal-reducing bacteria while serving to activate the metal-reducing bacteria. To this end, the electron donors may be one or more selected from organic acids and hydrogen gas. Here, the organic acids may be organic acids containing a carboxylic group, organic acids containing a sulfonic acid group, or mixed acids thereof. The organic acids containing the carboxylic group may be one or two or more selected from citric acid, succinic acid, tartaric acid, formic acid, oxalic acid, malic acid, malonic acid, benzoic acid, maleic acid, gluconic acid, glycolic acid, and lactic acid. The organic acids containing the sulfonic acid group may be one or two or more selected from methanesulfonic acid, ethanesulfonic acid, propanesulfonic acid, aminomethanesulfonic acid, benzenesulfonic acid, toluene sulfonic acid (4-methylbenzenesulfonic acid), sodium toluene sulfonate, phenolsulfonic acid, pyridinesulfonic acid, dodecylbenzene sulfonic acid, and methylphenolsulfonic acid.

In the method for removing cesium ions according to the exemplary embodiment of the present invention, concentrations of the electron donors are not limited as long as the electron donors may provide electrons to the sulfur species by the metal-reducing bacteria at the concentration, but may be specifically, 5 to 20 mM, and more specifically, 7 to 15 mM. In the case in which an amount of the added electron donors is below the above-mentioned range, it is impossible to sufficiently supply the electrons to the sulfur species, such that the new phase of mineral containing cesium may not be sufficiently formed, and in the case in which the amount of the added electron donors is over the above-mentioned range the formation of the mineral containing cesium may be hindered by them, and the electron donors may affect the formation rate of the mineral.

In the method for removing cesium ions according to the exemplary embodiment of the present invention, a pH of the solution in which the mineral containing cesium is formed may be 7.0 to 8.5, preferably, 7.3 to 8.0. In the case in which the pH of the solution is below the above-mentioned range, that is, acidic, Pautovite may be slowly formed, and in the case in which the pH is over the above-mentioned range, the solution is strongly basic, which may inhibit activity of the metal-reducing bacteria.

Therefore, in the method for removing cesium ions according to the exemplary embodiment of the present invention, in the case in which the pH of the solution containing the cesium ions is in the above-mentioned range, a separate pH adjusting step is not required, but in the case in which the pH of the solution containing the cesium ions is out of the above-mentioned range, the pH of the solution may be adjusted to be in the above-mentioned range by mixing a pH adjustment reagent with the solution. Here, as the pH adjustment reagent, any acidic or basic compound may be used without limitation as long as it may change the pH of the solution containing the cesium ions to be set in the above-mentioned range. In detail, the acids capable of being used as the pH adjustment reagent may be one or two or more selected from hydrochloric acid, sulfuric acid, nitric acid, hydrofluoric acid, boric acid, carbonic acid, hypophosphorous acid, phosphorous acid, phosphoric acid, formic acid, acetic acid, propionic acid, butyric acid, valeric acid, 2-methylbutyric acid, n-hexanoic acid, n-heptanoic acid, n-octanoic acid, benzoic acid, glycolic acid, salicylic acid, glyceric acid, oxalic acid, malonic acid, succinic acid, glutaric acid, adipic acid, pimelic acid, maleic acid, phthalic acid, malic acid, tartaric acid, citric acid, lactic acid, diglycolic acid, 2-furancarboxylic acid, methoxyacetic acid, methoxyphenylacetic acid, and the like, but is not limited thereto. The bases capable of being used as the pH adjustment reagent may be one or two or more selected from lithium hydroxide, sodium hydroxide, potassium hydroxide, magnesium hydroxide, calcium hydroxide, barium hydroxide, copper hydroxide, sodium carbonate, potassium carbonate, sodium bicarbonate, potassium bicarbonate, ammonia gas, ammonia water, methyl amine, trimethylamine, triethylamine, and the like, but is not limited thereto.

In the method for removing cesium ions according to the exemplary embodiment of the present invention, a removal reaction temperature of the cesium ions is not particularly limited as long as the metal-reducing bacteria may be active at the temperature, but the removal reaction temperature may be specifically 0 to 45° C., and more specifically, 20 to 35° C. In the case of removing the cesium ion in the temperature range described above, the cesium ions may be rapidly removed by activity of the metal-reducing bacteria.

The method for removing cesium ions according to an exemplary embodiment of the present invention includes:

mixing the sulfur source and the pH adjustment reagent with the solution containing the cesium ions (step a); and

adding the metal-reducing bacteria, an iron source, and the electron donors with the solution in step a (step b).

In the case of removing the cesium ions through two steps as described above, there is an advantage in that an influence by pH may be excluded as much as possible by injecting the bacteria into the solution of which the pH is adjusted. Further, in the case in which the sulfur source in step a is Na₂SO₃ or NaHSO₃, the sulfur source may be injected as a reducing agent capable of removing dissolved oxygen present in the solution.

In the method for removing cesium ions according to the exemplary embodiment of the present invention, during step a, a step of sterilizing the solution containing the cesium ions may be additionally performed. In the case of sterilizing the solution containing the cesium ions, a negative influence of other bacteria on the metal-reducing bacteria added into step b may be significantly blocked. A method for sterilizing the solution containing the cesium ions is not limited as long as the method is generally used for the sterilization of a solution. More specifically, the solution may be sterilized by applying ultra violet (UV) light, heat, or the like.

The present invention provides an apparatus for removing cesium ions.

In addition, the present invention provides an apparatus for removing cesium ions using the method for removing cesium ions described above.

Hereinafter, the apparatus for removing cesium ions will be described in detail with reference to the accompanying drawings. The accompanying drawings of the present invention are provided in order to more completely explain the present invention to those skilled in the art, and shapes, sizes, and the like, of components shown in the drawings may be simplified or exaggerated.

The apparatus for removing cesium ions according to the present invention may include an anaerobic tank into which a solution containing the cesium ions is introduced; and a microbial purification tank which is in connection with the anaerobic tank and into which the solution containing the cesium ions is introduced, wherein the anaerobic tank is supplied with a sulfur source and a pH adjustment reagent, and the microbial purification tank is supplied with metal-reducing bacteria, iron ions, and an electron donors. With the apparatus described above, the cesium ions may be effectively mineralized into Pautovite by the metal-reducing bacteria to thereby be precipitated, such that the cesium ions may be removed as sludge with a compact volume.

In the case of removing cesium ions using the apparatus for removing cesium ions according to the present invention, there are advantages in that the cesium ions may be efficiently removed using a significantly simple apparatus, cesium may be selectively removed even in a solution in which competing ions are present, such as sea water, cesium ions may be removed with high efficiency, even with a low concentration at which it is difficult to remove cesium ions, and since an amount of wastes formed after removing the cesium ions is very small, a high expense for disposing wastes is not required.

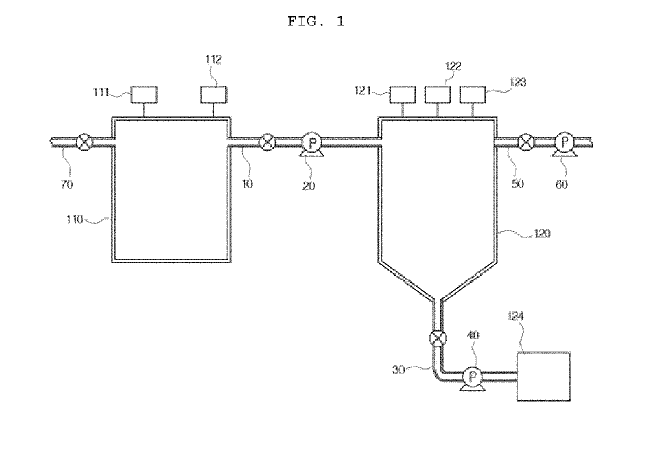

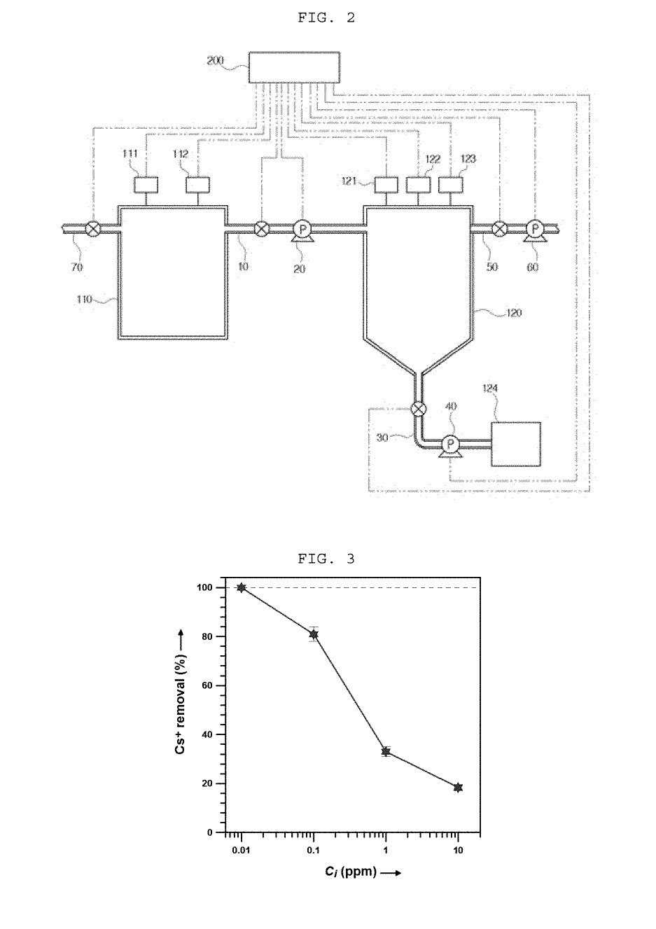

FIG. 1 is a schematic view illustrating an apparatus for removing cesium ions according to an exemplary embodiment of the present invention. As illustrated in FIG. 1, the apparatus for removing cesium ions may include an anaerobic tank 110 and a microbial purification tank 120 which is in connection with the anaerobic tank. In detail, the anaerobic tank may be provided in the front of the microbial purification tank based on a flow of solution containing cesium ions.

In the apparatus for removing cesium ions according to the present invention, the anaerobic tank adjusts a pH of the introduced solution containing the cesium ions and then supplies the solution containing the cesium ions to the microbial purification tank. Additionally, after sterilizing the solution containing the cesium ions and removing dissolved oxygen in the solution containing the cesium ions in the anaerobic tank, the anaerobic tank may supply the solution from which the dissolved oxygen is removed to the microbial purification tank.

To this end, the anaerobic tank may include a pH adjustment reagent storage tank 112 and a sulfur source storage tank 111, and be connected to the pH adjustment reagent storage tank 112 and the sulfur source storage tank 111 through openable and closable connecting pipes, respectively.

Acid or base reagents for adjusting a pH in the anaerobic tank to 7 to 8.5 may be stored in the pH adjustment reagent storage tank. The acids capable of being used as the pH adjustment reagent may be one or two or more selected from hydrochloric acid, sulfuric acid, nitric acid, hydrofluoric acid, boric acid, carbonic acid, hypophosphorous acid, phosphorous acid, phosphoric acid, formic acid, acetic acid, propionic acid, butyric acid, valeric acid, 2-methylbutyric acid, n-hexanoic acid, n-heptanoic acid, n-octanoic acid, benzoic acid, glycolic acid, salicylic acid, glyceric acid, oxalic acid, malonic acid, succinic acid, glutaric acid, adipic acid, pimelic acid, maleic acid, phthalic acid, malic acid, tartaric acid, citric acid, lactic acid, diglycolic acid, 2-furancarboxylic acid, methoxyacetic acid, methoxyphenylacetic acid, and the like, but is not limited thereto. The bases capable of being used as the pH adjustment reagent may be one or two or more selected from lithium hydroxide, sodium hydroxide, potassium hydroxide, magnesium hydroxide, calcium hydroxide, barium hydroxide, copper hydroxide, sodium carbonate, potassium carbonate, sodium bicarbonate, potassium bicarbonate, ammonia gas, ammonia water, methyl amine, trimethylamine, triethylamine, and the like, but is not limited thereto. In detail, the above-mentioned pH adjustment reagent may be stored in the pH adjustment reagent storage tank, or a solution in which the pH adjustment reagent is dissolved may be stored in the pH adjustment reagent storage tank, but is not limited as long as it may supply the pH adjustment reagent.

The sulfur source storage tank may supply the sulfur source to the solution containing the cesium ion, stored in the anaerobic tank. The sulfur source may be reduced by the metal-reducing bacteria in the microbial purification tank to thereby be converted into a sulfide form incorporating cesium together with iron ions. In detail, the sulfur source is not limited as long as it is a compound capable of supplying anions represented by SO₄²⁻, SO₃²⁻, SO₂²⁻, S₂O₃²⁻, S₂O₄²⁻, S₂O₅²⁻, S₂O₆²⁻, S₂O₇²⁻, S₂O₈²⁻, S₄O₇²⁻, or S₄O₆²⁻ in a solution, but the sulfur source may be specifically Na₂SO₃, NaHSO₃, or Na₂SO₄, and more specifically Na₂SO₃ or NaHSO₃.

In the case in which the sulfur source described above is Na₂SO₃ or NaHSO₃, the sulfur source may also remove the dissolved oxygen in the solution containing the cesium ions. In detail, in the case in which Na₂SO₃ or NaHSO₃ is dissolved in the solution, SO₃²⁻ may be formed, and then SO₃²⁻ may react with the dissolved oxygen to form SO₄²⁻ removing the oxygen from solution.

The anaerobic tank may further include a general stirring device for uniformly mixing the solution containing the cesium ions, and further include a sterilizing device for sterilizing the solution that may include other bacteria. Here, the sterilizing device is not limited as long as it is a general device used for the sterilization of solution containing the cesium ions, but the sterilizing device may be specifically a UV sterilizing device. Further, the anaerobic tank may be a closed reaction tank to prevent the dissolution of oxygen from the atmosphere and the leakage of radionuclides, but is not limited thereto.

The above-mentioned anaerobic tank may be in connection with the microbial purification tank, and a connecting pipe 10 between the anaerobic tank and the microbial purification tank may be openable and closable and further include pump 20 for transferring the solution containing the cesium ions.

In the microbial purification tank 120, the cesium ions in the solution containing the cesium ions, which is supplied to the microbial purification tank in a state in which the pH thereof is adjusted and oxygen is removed, are converted into the sulfide mineral containing cesium to thereby be removed. In detail, the sulfide mineral containing the cesium ions becomes sludge to thereby be easily separated from the solution. To this end, the microbial purification tank may have a tapered shape of which a lower portion becomes gradually narrow, in order to separate the precipitated sludge and purified water from which the cesium ions are separated. Here, the tapered shape of the lower portion of the microbial purification tank may include a cone shape.

In the apparatus for removing cesium ions according to the exemplary embodiment of the present invention, the microbial purification tank may include an iron source storage tank 121, a metal-reducing bacteria storage tank 122, and an electron donor storage tank 123 in order to convert the cesium ion into the mineral form containing cesium to effectively separate the cesium ions, and the microbial purification tank may be in connection with the iron source storage tank, the metal-reducing bacteria storage tank, and the electron donor storage tank through openable and closable pipes, respectively. Further, the microbial purification tank may include a general stirring device for uniformly mixing the solution containing the cesium ions.

The iron source storage tank supplies iron (II) ions to the microbial purification tank. As the iron source, any compound may be used without limitation as long as it may provide divalent iron ions to the solution. In detail, the iron source may be one or two or more selected from iron (II) chloride, iron (II) sulfate, iron (II) acetate, iron (II) bromide, and iron (II) nitride. The iron ions supplied to the microbial purification tank may be bound to the cesium ions and the sulfur source reduced by the metal-reducing bacteria to thereby be converted into the sulfide mineral containing cesium. The iron source stored in the iron source storage tank may be in a form of the above-mentioned iron source or a solution in which the iron source is dissolved, but the form of the iron source is not limited as long as the iron source may be supplied to the microbial purification tank.

The metal-reducing bacteria storage tank supplies the metal-reducing bacteria to the microbial purification tank. The metal-reducing bacteria react with the sulfur source to form S²⁻ in the microbial purification tank, and the formed S²⁻, the cesium ions, and the iron ions may react together to thereby be converted into the sulfide mineral containing cesium, specifically, Pautovite. As the metal-reducing bacteria, any bacteria may be used without limitation as long as they may convert the sulfur source into S²⁻, but the metal-reducing bacteria may be one or two or more selected from Pseudomonas, Shewanella, Clostridiums, Desulfovibrio, Desulfosporosinus, Desulfotomaculum, Anaeromyxobacter, and Geobacters.

A metal-reducing bacteria powder or a cultured solution containing the metal-reducing bacteria may be stored in the metal-reducing bacteria storage tank, but a form of the metal-reducing bacteria is not limited as long as the metal-reducing bacteria may be supplied to the microbial purification tank.

The electron donor storage tank may supply the electron donors to the microbial purification tank. The electron donors may activate the metal-reducing bacteria and provide electrons required for the reduction of sulfur oxyanions. In detail, the electron donors may be one or two or more selected from hydrogen gas, citric acid, succinic acid, tartaric acid, formic acid, oxalic acid, malic acid, malonic acid, benzoic acid, maleic acid, gluconic acid, glycolic acid, lactic acid, methanesulfonic acid, ethanesulfonic acid, propanesulfonic acid, aminomethanesulfonic acid, benzenesulfonic acid, toluene sulfonic acid (4-methylbenzenesulfonic acid), sodium toluene sulfonate, phenolsulfonic acid, pyridinesulfonic acid, dodecylbenzene sulfonic acid, and methylphenolsulfonic acid. In the case in which the electron donor is hydrogen gas, the electron donor contained in the electron donor storage tank may be in a form of pure hydrogen gas or a mixture of hydrogen gas and one or two or more gases selected from nitrogen, argon, neon, and helium. Further, in the case in which the electron donors are one or two or more selected from the above-mentioned organic acids, the electron donors may be stored in a form of the electron donor itself or a solution of the electron donors, but is not limited thereto.

The cesium ions in the solution may be converted into the mineral containing cesium in the microbial purification tank, and the mineral containing cesium may be precipitated in a form of sludge. Therefore, the lower portion of the microbial purification tank may be provided with sludge discharge pipe 30, which is an openable and closable pipe for discharging the sludge, and the precipitated sludge may be transferred to a sludge storage tank 124 through the sludge discharge pipe. Further, a sludge dehydration tank for removing water remaining in the sludge may be further provided in the front of the sludge storage tank, and the dehydrated sludge may be stored in the sludge storage tank.

In addition, an openable and closable purified water discharge pipe 50 may be connected to the microbial purification tank, and the purified water from which the cesium ions are removed may be discharged through the purified water discharge pipe. Here, in the discharged purified water, the cesium ions are removed, and the discharged purified water is weakly alkaline, such that the purified water may be directly discharged without a post treatment.

The apparatus for removing cesium ions according to the exemplary embodiment of the present invention may further include a control part 200.

In detail, the control part may control an openable and closable cesium ion-containing solution inflow pipe 70 connected to the anaerobic tank, to adjust whether or not to introduce solution containing cesium ions and adjust an amount of the solution containing cesium ions in the anaerobic tank, and may control a first transfer pipe 10 and a first transfer pump 20 to control whether or not to transfer the solution containing cesium ions from the anaerobic tank to the microbial purification tank. After a predetermined amount of solution containing the cesium ions is introduced into the anaerobic tank by the control part, the control part may control transfer pipes and pumps so that predetermined amounts of the sulfur source and the pH adjustment reagent are injected from the sulfur source storage tank and the anaerobic reagent storage tank to the anaerobic tank, respectively.

After the oxygen in the solution containing the cesium ions is removed in the anaerobic tank, the control part may control the first transfer pipe and the first transfer pump to move the solution containing cesium ions from the anaerobic tank to the microbial purification tank. Thereafter, the control part may control opening and closing of transfer pipes and operations of pumps so that predetermined amounts of the iron source, the metal-reducing bacteria, and the electron donors are injected from the iron source storage tank 121, the metal-reducing bacteria storage tank 122, and the electron donor storage tank 123 to the microbial purification tank, respectively.

After the cesium ions are precipitated in a form of the sludge by the metal-reducing bacteria in the microbial purification tank, and purification of the solution containing cesium ions is completed, the control part may control the sludge discharge pipe 30 and a sludge discharge pump 40 to separate and discharge the sludge precipitated in the lower portion of the microbial purification tank. Thereafter, the control part may control the purified water discharge pipe 50 and a purified water discharge pump 60 to discharge purified water from which cesium ions are removed.

[Measurement of Cesium Ion Removal Efficiency in Sea Water]

Sea water samples in which 0.01 ppm, 0.1 ppm, 1 ppm, and 10 ppm of cesium ions were contained were prepared, respectively, and an anaerobic tank and a microbial purification tank were provided. A pH of each of the sea water samples containing the cesium ions was adjusted to 7.5 by mixing NaHCO₃ and HCl with the sea water sample in the anaerobic tank, and sodium sulfite was added thereto as a sulfur source. Here, an amount of added sodium sulfite was 10 g based on 10 kg of a solution containing the cesium ions. After the mixture was stirred for 12 hours in the anaerobic tank, the solution containing the cesium ions was transferred to the microbial purification tank. Iron (II) chloride as an iron source, lactic acid as an electron donor, and Desulfovibrio vulgaris as metal-reducing bacteria were mixed with the solution containing the cesium ions in the microbial purification tank. Here, iron chloride, lactic acid, and Desulfovibrio vulgaris were mixed so as to have concentrations of 1 mM, 10 mM, and 1.0 mg/L (based on a protein concentration), respectively. A reaction was carried out for 48 hours or more while stirring each of the solutions containing the cesium ions in the microbial purification tank, and precipitated Pautovite was separated. Then, purified water was collected, and a concentration of the cesium ions therein was measured. The result was shown in FIG. 3.

Referring to FIG. 3, it may be unusual that the efficiency of cesium removal interestingly increased while the concentration of the cesium ions decreased, and even in the case in which the concentration of the cesium ions in the sea water sample was 0.01 ppm or less, the removal efficiency was improved to 99%.

[Measurement of Cesium Ion Removal Efficiency in Fresh Water]

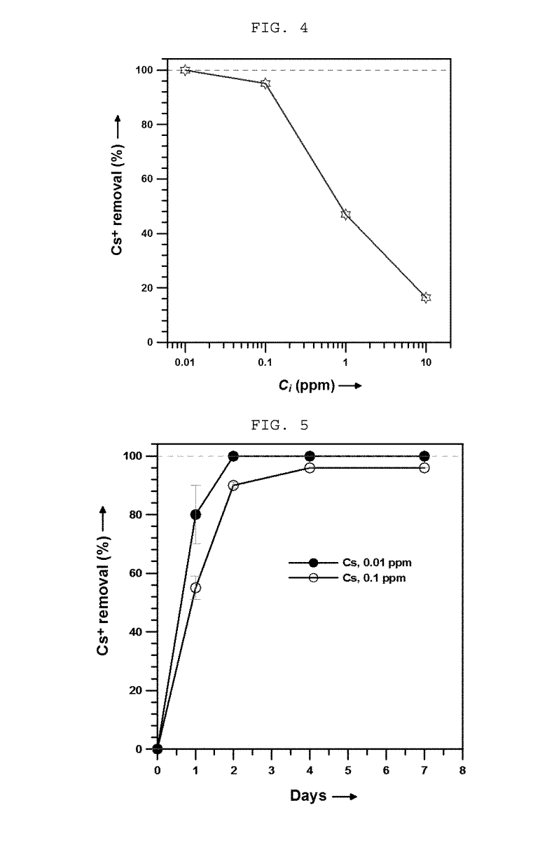

Cesium ion removal efficiency was measured by the same method for fresh water instead of sea water, and the result was shown in FIG. 4.

Referring to FIG. 4, it may be also unusual that the efficiency of cesium removal interestingly increased while the concentration of the cesium ions decreased, and even in the case in which the concentration of the cesium ions was 0.01 ppm or less, the removal efficiency was improved to 99%.

[Measurement of Cesium Ion Removal Efficiency with Passage of Time]

Cesium ion removal efficiency for fresh water was measured, in the microbial purification tank at the cesium concentrations of 0.01 ppm and 0.1 ppm with time. The result was shown in FIG. 5.

Referring to FIG. 5, Most of cesium (90% or more) was removed from the solution after 48 hours.

[Confirmation of Mineral Containing Cesium]

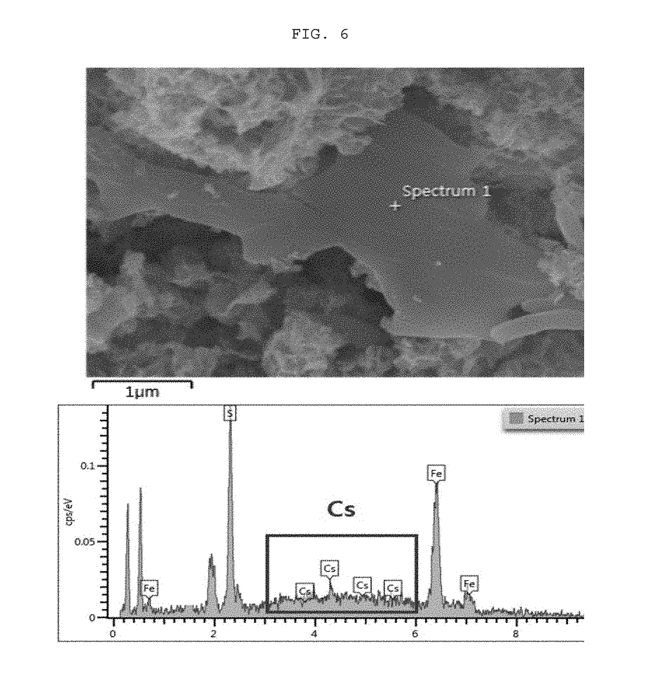

After removing cesium ions from sea water, the formed sludge was separated and analyzed using scanning electron microscope (FESEM, S-4700, Hitachi). The obtained FESEM photograph and element analyses results are exhibited in FIG. 6.

Referring to FIG. 6, a crystalline mineral phase incorporating cesium was formed and grew to a size of μm scale in the sludge.

[Confirmation of Formation of Pautovite]

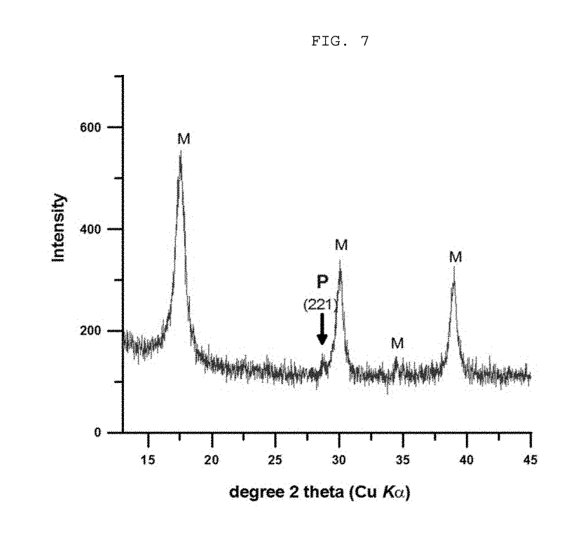

After measuring cesium ion removal efficiency in sea water, the formed sludge was separated and analyzed using X-ray diffraction analysis (Bruker D8. Advance diffractometer), and the result was illustrated in FIG. 7.

Referring to FIG. 7, Pautovite crystalline mineral was evidently formed along with Mackinawite (FeS) showing the Pautovite's main peak (221: Miller indices (hkl)).

The method and the apparatus for removing cesium ions according to the present invention have an advantage in that a large amount of cesium ions may be efficiently removed at room temperature.

The method and the apparatus for removing cesium ions according to the present invention have an advantage in that the cesium ions may be removed with high efficiency even at a low concentration at which it is difficult to remove the cesium ions other methods.

The method and the apparatus for removing cesium ions according to the present invention have an advantage in that the cesium ions may be removed with high efficiency even in the case (for example, sea water condition) in which competing ions are present at high concentrations.

The method and the apparatus for removing cesium ions according to the present invention have an advantage in that since the cesium ions are compacted in a solid form of crystalline mineral, a volume of the wastes is significantly small.

In the case of using the method and the apparatus for removing cesium ions according to the present invention, there is an advantage in that since a form of the formed waste is the crystalline mineral, long-term disposal stability is high in underground.

What is claimed is:

1. A method for removing cesium ions, the method comprising: adding metal-reducing bacteria, an iron source, and a sulfur source into a solution containing the cesium ions to convert the cesium ions into a solid mineral incorporating cesium.

2. The method of claim 1, wherein the mineral incorporating cesium is Pautovite (CeFe₂S₃).

3. The method of claim 1, wherein in the converting of the cesium ions into the mineral incorporating cesium, a pH of the solution is 7 to 8.5.

4. The method of claim 1, wherein the metal-reducing bacteria are one or two or more selected from the group consisting of Pseudomonas, Shewanella, Clostridium, Desulfovibrio, Desulfosporosinus, Desulfotomaculum, Anaeromyxobacter, and Geobacter.

5. The method of claim 1, wherein a concentration of the metal-reducing bacteria (based on a protein concentration) is 0.3 to 5 mg/L.

6. The method of claim 1, wherein the iron source is one or two or more selected from iron (II) chloride, iron (II) sulfate, iron (II) acetate, iron (II) bromide, and iron (II) nitride.

7. The method of claim 1, wherein a concentration of the iron source is 0.5 to 5 mM.

8. The method of claim 6, wherein the sulfur source is a compound forming anions represented by SO₄²⁻, SO₃²⁻, SO₂²⁻, S₂O₃²⁻, S₂O₄²⁻, S₂O₅²⁻, S₂O₆²⁻, S₂O₇²⁻, S₂O₈²⁻, S₄O₇²⁻, or S₄O₆²⁻.

9. The method of claim 1, wherein the sulfur source is a dissolved oxygen scavenger.

10. The method of claim 1, wherein a concentration of the sulfur source is 0.3 to 2.0 mM.

11. The method of claim 1, wherein an electron donor is additionally injected into the solution containing the cesium ions.

12. The method of claim 1, wherein a concentration of cesium in the solution containing the cesium ions is 0.5 ppm or less.

13. The method of claim 1, wherein the solution containing the cesium ions is sea water.

14. An apparatus for removing cesium ions, the apparatus comprising:
an anaerobic tank into which a solution containing cesium ions is introduced and to which a sulfur source and a pH adjustment reagent are supplied; and
a microbial purification tank that is in connection with the anaerobic tank and to which metal-reducing bacteria, an iron source, and an electron donor are supplied,
wherein cesium ions are converted into a solid mineral incorporating cesium by the activity of metal-reducing bacteria to thereby be precipitated in the microbial purification tank, such that the cesium ions in the solution containing cesium ions are removed in a form of compact sludge.

15. The apparatus of claim 14, further comprising:
a first transfer pipe connecting the anaerobic tank and the microbial purification tank so as to be openable and closable;
a first transfer pump connected to the first transfer pipe to transfer the solution containing cesium ions in the anaerobic tank to the microbial purification tank;
a sludge discharge pipe installed so as to be in connection with a lower portion of the microbial purification tank and be openable and closable; and
a sludge discharge pump connected to the sludge discharge pipe to discharge the sludge accumulated from the microbial purification tank.
